(19) United States
(12) Patent Application Publication
(10) Pub. No.: US 2026/0064887 A1
(43) Pub. Date: Mar. 5, 2026

(54) METHODS AND SYSTEMS FOR STORING AND CONTROLLING ACCESS TO PROTECTED HEALTH DATA, MAKING DE-IDENTIFIED HEALTH DATA AVAILABLE FOR USE AND RESOLVING IDENTITIES OF DE-IDENTIFIED HEALTH DATA TO AUTHORIZED USERS

(71) Applicant: KONINKLIJKE PHILIPS N.V., EINDHOVEN (NL)

(72) Inventors: Man Ching Cheung, Cambridge, MA (US); JONATHAN KAMERMAN, Andover, MA (US)

(21) Appl. No.: 19/293,226

(22) Filed: Aug. 7, 2025

Related U.S. Application Data
(60) Provisional application No. 63/690,446, filed on Sep. 4, 2024.

(51) Int. Cl. G06F 21/62; G06F 21/60

(52) U.S. Cl. CPC G06F 21/6254; G06F 21/602

(57) ABSTRACT

A method for storing and controlling access to protected health information (PHI) data, comprising: obtaining health data, wherein the health data comprises, for at least some of a plurality of subjects, use right authorization received from the subject; removing identifying information from the health data to generate de-identified health data and PHI data; encrypting the PHI data; storing the encrypted PHI data in a patient data database, wherein the stored encrypted PHI data for each subject is associated with: (i) a unique subject token for that subject; (ii) a use right authorization received from that subject; and (iii) a corresponding access token for that subject; receiving, from a requester, a request for access to health data; determining which stored encrypted PHI data can be accessed by the requester; and providing access to only the health data for which the requester is determined to be authorized to access.

### FIELD OF THE DISCLOSURE

The present disclosure is directed generally to methods and systems for storing and controlling access to protected health information (PHI), providing de-identified data for analysis and insights, and enabling recombining PHI with de-identified health data, for a plurality of subjects.

### BACKGROUND

Patient health data is an enormously valuable commodity. This data is utilized for a wide variety of purposes, including standard of care and clinical quality analysis, research, algorithm development and testing, third-party tool development and testing, artificial intelligence development and testing, and many more. Some of the challenges with delivering insights from patient health data as a product or service include the definition of bill of materials, data access control, and scope creep. From a customer perspective, insight is about transforming data to make visible information, so users' experience and knowledge can be engaged in a meaningful way. In practice, what this means is that analysis is an active and dynamic process. By necessity, then, the questioning aspect of data analysis and insights is free flowing and wide ranging. As a result, there is difficulty in creating a digital data product, or writing a professional services contract, that can accommodate this type of usage, let alone finding the right business model to deliver.

Current approaches to analytics rely on providing services where technical experts create ad-hoc data models, reports, and dashboards. While this process can be part of a services-based business model, the heavy technical lift generally means that considerable billable hours are spent on solution development deployment rather than on generating insights with customers. In addition, the nature of this work means that analysts generally have access to patient health information (PHI). Thus, these approaches are expensive and elevate the risk of breach of protected information.

### SUMMARY OF THE DISCLOSURE

There is thus a continued need for methods and systems that enable access to de-identified patient health data in a more efficient and affordable manner. Two systems are described. The first system is a standard keyed architecture, whereby specific modules (data architecture) are enabled based on the purchase of data consultancy or analytics products and services. When a proper access key—linked to a sales option—is purchased, the set of algorithms to create the data architecture is activated. The data architecture is designed to remove protected health information (PHI). A key insight is that for nearly all use cases (analytics or research), PHI is rarely needed. However, for clinically oriented research or normal clinical workflow, clinicians and clinician-scientists may in fact require granular detail involving PHI. A second system is engaged in order to govern and control access and use rights of patients' PHI data.

Various embodiments and implementations are directed to a method and system for storing and controlling access to de-identified health data using a health data storage and access system. The system receives health data for a plurality of subjects, comprising use right authorization received from those subjects. The system removes identifying information from the health data to generate de-identified health data, but retains an access token that identifies the original health data for that subject. The PHI data from the source is encrypted and stored in a patient data database, while the encrypted stored health data for each subject is associated with: (i) a unique subject token for that subject; (ii) the use right authorization received from that subject; and (iii) encryption and decryption keys for the patient; (iv) the corresponding access token for that subject based on the originating study; and (v) for uses not related to the direct provision of healthcare to further limit the manner in which PHI is presented, such as removing identifiers and providing categorized/aggregated PHI where re-identification cannot occur by means reasonably likely to be used. To efficiently access the stored data the system receives, from a requester having a unique requester access key, a request for access to the stored encrypted identified health data. The system determines, using the unique requester access key and the use right authorizations received from the subjects, whether the encrypted PHI data can be decrypted and then matched by the access token, with the proper use case allowed by the patient for the requester. The system then provides, based on the determination, access to only the stored encrypted PHI for which the requester is determined to be authorized to access.

According to an aspect, a method for storing and controlling access to protected health information (PHI) data for a plurality of subjects is provided. The method includes: obtaining health data for the plurality of subjects, wherein the health data comprises one or more data elements for each of the plurality of subjects, and wherein the health data comprises, for at least some of the plurality of subjects, use right authorization received from the subject; removing identifying information from the health data to generate de-identified health data and PHI data, wherein the de-identified health data for each subject is associated with an access token that identifies the original health data for that subject; encrypting the PHI data; storing the encrypted PHI data in a patient data database, wherein the stored encrypted PHI data for each subject is associated with: (i) a unique subject token for that subject; (ii) the use right authorization received from that subject; and (iii) the corresponding access token for that subject; receiving, from a requester, a request for access to the stored encrypted PHI data, wherein the requester comprises a unique requester access key; determining, based on the unique requester access key and the use right authorizations received from the subjects, which stored encrypted PHI data can be accessed by the requester; and providing, to the requester based on the determination, access to only the stored encrypted PHI data for which the requester is determined to be authorized to access.

According to an embodiment, the encrypted PHI data for each subject is stored as a plurality of data elements, each of the plurality of data elements associated with (i) the unique subject token for that subject; (ii) the use right authorization received from that subject; and (iii) the corresponding access token for that subject.

According to an embodiment, the method further includes: receiving, from a subject, use right authorization for the subject's health data.

According to an embodiment, the subject accesses a remote portal to provide the use right authorization.

According to an embodiment, the method further includes: receiving, from a requester, a request for the original health data or PHI data for one or more subjects; determining that the requester has authorization to access the original health data for the one or more subjects; providing, using the access token associated with each of the one or more subjects, access to the original health data for the one or more subjects for a use case related to healthcare delivery; and/or providing, using the access token associated with each of the one or more subjects, limited and aggregated PHI data for the one or more subjects, for a use case not related to healthcare delivery.

According to an embodiment, the use right authorization received from the subject comprises which data points in the health data for that subject may be used.

According to an embodiment, the use right authorization received from the subject comprises one or more uses for which that health data may be utilized.

According to an embodiment, the use right authorization received from the subject comprises an identification of one or more entities authorized to use the subject's health data.

According to another aspect is a system for storing and controlling access to protected health information (PHI) data for a plurality of subjects. The system includes: health data for the plurality of subjects, wherein the health data comprises one or more data elements for each of the plurality of subjects, and wherein the health data comprises, for at least some of the plurality of subjects, use right authorization received from the subject; and a processor configured to: (i) remove identifying information from the health data to generate de-identified health data and PHI data, wherein the PHI data for each subject is associated with an access token that identifies the original health data for that subject; (ii) encrypt the PHI data; (iii) store the encrypted de-identified health data in a patient data database, wherein the stored encrypted PHI data for each subject is associated with: (1) a unique subject token for that subject; (2) the use right authorization received from that subject; and (3) the corresponding access token for that subject; (iv) receive, from a requester, a request for access to the stored encrypted PHI data, wherein the requester comprises a unique requester access key; (v) determine, based on the unique requester access key and the use right authorizations received from the subjects, which stored encrypted PHI data can be accessed by the requester; and (vi) provide, to the requester based on the determination, access to only the stored encrypted PHI data for which the requester is determined to be authorized to access.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1A is a flowchart of a method for storing and controlling access to de-identified health data for a plurality of subjects, in accordance with an embodiment.

FIG. 1B is a flowchart of a method for storing and controlling access to de-identified health data for a plurality of subjects, in accordance with an embodiment.

FIG. 2 is a schematic representation of a health data storage and access system, in accordance with an embodiment.

FIG. 3 is a flowchart of a method for storing and controlling access to de-identified health data for a plurality of subjects, in accordance with an embodiment.

### DETAILED DESCRIPTION OF EMBODIMENTS

The present disclosure describes various embodiments of a system and method configured to store encrypted PHI data and control access to de-identified health data using a health data storage and access system. More generally, Applicant has recognized and appreciated that it would be beneficial to enable access to de-identified patient health data in a more efficient and affordable manner. Thus, a health data storage and access system receives health data for a plurality of subjects, optionally from a plurality of data sources, comprising use right authorization received from those subjects. The system removes identifying information from the health data to generate de-identified health data, but retains an access token that identifies the original health data for that subject. The original identified health data (e.g., PHI data) is encrypted and stored in a patient data database (that is, a “patient vault”), and the stored encrypted PHI data for each subject is associated with: (i) a unique subject token for that subject; (ii) the use right authorization received from that subject; and (iii) the corresponding encryption and access token(s) for that subject. To efficiently access the stored data the system receives, from a requester having a unique requester access key, a request for access to the stored encrypted PHI data. The system determines, using the unique requester access key and the use right authorizations received from the subjects, which stored encrypted PHI data can be accessed by the requester. The system then provides, based on the determination, access to only the stored encrypted PHI data, or to a limited, aggregated set of PHI data for which the requester is determined to be authorized to access.

According to an embodiment, the embodiments and implementations disclosed or otherwise envisioned herein use a token-based approach that activates data integration algorithms (commonly referred to as “extract, transform, and load” or “ETL”) on the fly and builds a set of data models that can be connected or plugged-in in any sequence or order. In other words, the underlying data product is configured based on common customer needs rather than arbitrary constraints in terms of digital product architecture.

Further, the embodiments and implementations disclosed or otherwise envisioned herein prevent unnecessary or unauthorized access to PHI. Although many entities require patient health data for things such as research and quality care improvement, they typically do not need the associated PHI. The systems thus provide additional value for healthcare providers and patients; not only do they simplify the data structure and de-identify data, they create a set of data usage tokens that together form a description of data-use rights as allowed by the user. In addition to access to PHI, use of the data at the level of each record is also specified, such that a secondary check can be made to allow or deny the use of the record for purposes aside from direct care—for example, but not limited to, uses such as academic research or AI model development and training. The access portal used to access data can encode the use of the data, thereby allowing the system to check the use against the patient specified authorizations. According to an embodiment, the first property specified in this token is a URL link back to the source diagnostic platform (and access controlled by the platform), so that properly credentialed and privileged users (including but not limited to healthcare providers) can always access the patient record needed to provide care.

Thus, presented herein are methods and systems to leverage encryption tokens in two ways: 1) create a keyed data infrastructure that is tied directly to unlocking specific sales options to implement ETL and compute modules for delivering scalable analytics; and 2) create a method to store patient tokens that contain parameters to link to source data and specify data use rights.

The resulting data structures are built on the fly, as data blocks, each self-sufficient and with unique identifiers to enable interlocking joins of each component. Such an analytics approach allows for uncoupling the insight platform from clinical platform data architectures. The keyed modules essentially clean the data for customers by reorganizing source data and algorithm-generated results. Each module creates a set of data blocks, which fit together to provide synergistic expansion of available insights. However, the record level tokens will allow authorized users to resolve PHI clinical platforms, based upon consented upon use cases, casing the workflow between insights and the source patient records.

A token-based data model enables the data pipes and data structure and addresses three broad categories of uses: operations, clinical search, and formatting the data for clinical science research. Rather than exposing all the source data to the customer, the embodiments described herein separate PHI from data (that is, de-identifying health data), generating data blocks for the insight platform, and where necessary enables authorized users to resolve granular patient information for authorized uses. For the cases where PHI is needed (by an authorized clinician-researcher user), they can get access back to the source record because the system stores an access token within the database. This allows for patient records to be used for basic operational improvements without PHI. Clinicians will always be able to resolve the original record from the diagnostic platform. For approved clinical research uses, those records can also be accessed within the research component of the insight platform.

Having a defined technical infrastructure also improves digital platform monetization. For example, new features can be placed by “sale option.” The marginal cost for delivery can be assessed and controlled, helping to classify new features by option or to establish new options. The modular framework enables cost structuring of data science-as-a-service approach and for controlled integration of learnings from the field into product.

According to an embodiment, the access portal used to access de-identified health data will comprise use tokens. The use tokens will be matched with the use right authorizations approved by the patient. Only when the portal and patient use right authorizations match will PHI be made available. For example, a clinical diagnosis portal will require that the PHI for a patient match with medical record numbers, name, date of birth, and/or other patient identifiers, and access to the PHI will be limited by privileges given to the user. As another example, a population health portal will have access to health data and limited PHI only for patients who consented to use of their data in this manner. As another example, a cohort selection tool for AI or ML training will only show patient health data and limited PHI for patients who consent to the use of their data in this manner.

According to an embodiment, for uses that are not directly related to providing care or diagnoses, the system will return a limited set of PHI. For non-healthcare delivery uses, parameters such as patient identifiers (e.g., home address, name, identification numbers, date of birth) will not be displayed. When PHI is combined with de-identified data, only a limited set of PHI data elements will be shown—for example, gender, race/ethnicity, and age, among others. For numerical PHI data that will be accessed, the data will be categorized and aggregated (that is, “binned”) to reduce risk of re-identification.

The embodiments and implementations disclosed or otherwise envisioned herein can be implemented with any system or process that may utilize or benefit from data with restricted or regulated data elements. For example, the embodiments and implementations disclosed or otherwise envisioned herein can be utilized to process and control health or not-health data from existing databases, and/or can be utilized to generate a new system or service that grants controlled access to patient health data. However, the disclosure is not limited to these systems or services, and thus the disclosure and embodiments disclosed herein can encompass any system that may utilize or benefit from controlled access to both restricted data (for example PHI data) and de-identified data (for example health data), or any system that requires data to be de-identified and lowering risk for algorithmically reconstructing the originating identity.

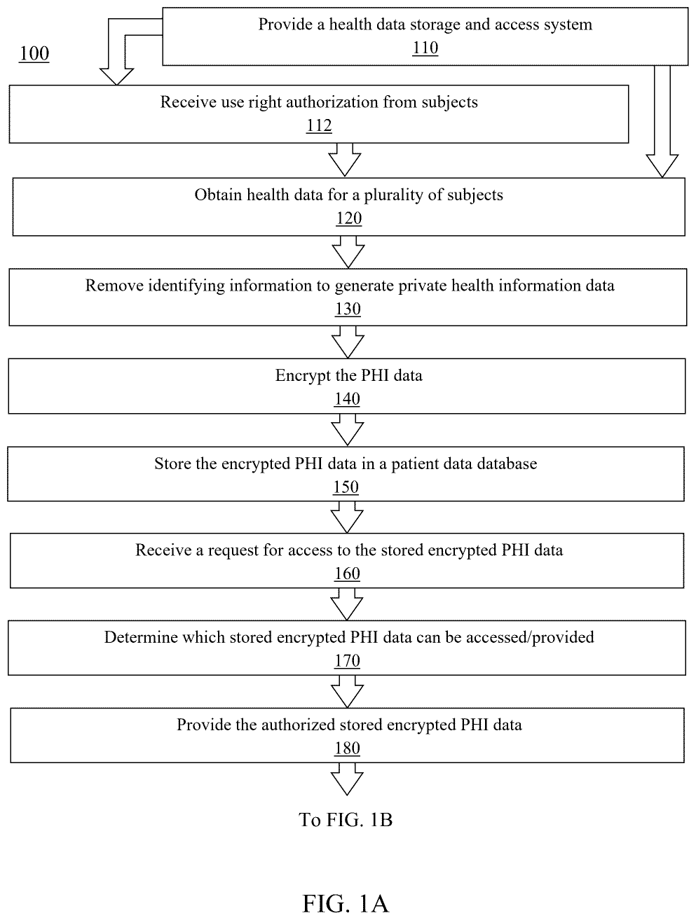

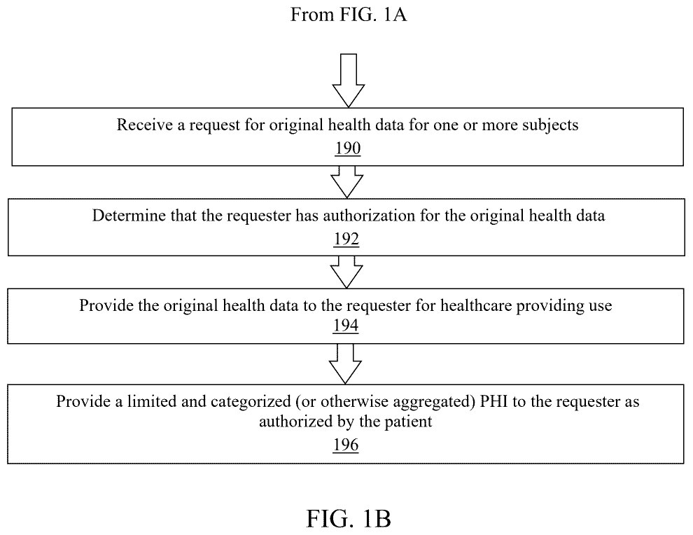

Referring to FIGS. 1A and 1B, in one embodiment, is a flowchart of a method 100 for storing and controlling access to de-identified health data for a plurality of subjects, using a health data storage and access system. The methods described in connection with the figures are provided as examples only, and shall be understood not to limit the scope of the disclosure. The health data storage and access system can be any of the systems described or otherwise envisioned herein. The health data storage and access system can be a single system or multiple different systems.

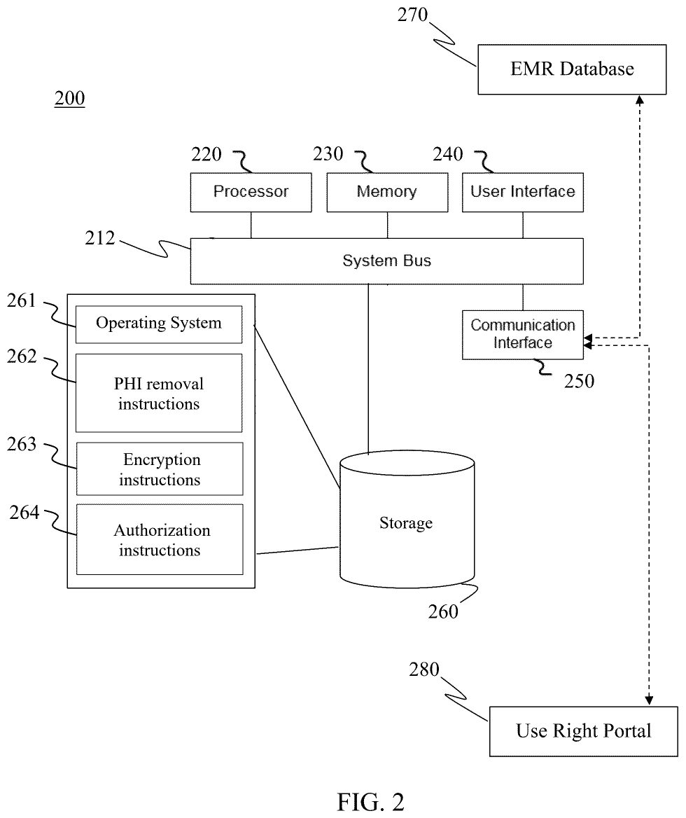

At step 110 of the method, a health data storage and access system 200 is provided. Referring to an embodiment of a health data storage and access system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, health data storage and access system 200 can be any of the systems described or otherwise envisioned herein. Health data storage and access system 200 can embody any system that is used to store health data with and/or without PHI data elements. Other elements and components of the health data storage and access system 200 are disclosed and/or envisioned elsewhere herein.

According to an embodiment, the health data storage and access system 200 comprises or is in direct or indirect communication with an electronic medical record (EMR) database or system 270, comprising health data for a plurality of patients or subjects. The health data can be any information about the plurality of patients or subjects. According to an embodiment, the information comprises one or more of demographic information about the patient, a diagnosis for the patient, medical history of the patient such as treatment information, and/or any other information. For example, demographic information may comprise information about the patient such as name, age, body mass index (BMI), and any other demographic information. The diagnosis for the patient may be any information about a medical diagnosis for the patient, historical and/or current. The medical history of the patient may be any historical admittance or discharge information, historical treatment information, historical diagnosis information, historical exam or imaging information, and/or any other information (although in some embodiments, a patient's medical history may not be available). Other patient information that can be received by the training system includes lab test results. For example, the lab tests may be an analysis of blood gases, electrolytes, biomarkers, and/or any other types of lab tests. Yet another example of patient information received by system 200 includes vital sign information for the patient. The vital sign information can be any vital sign of the patient such as heart rate, respiration rate, blood pressure, temperature, and/or any other information. Many other forms and types of patient health data are possible.

At optional step 112 of the method, which can happen at multiple different times within the method (including before and/or after step 120 of the method described below) the health data storage and access system 200 receives, from a subject, use right authorization for the subject's health data, for a specific event in time that gives rise to the health data. The use right authorization controls one or both of: (i) which data points in the health data for that subject may be used; and (ii) the uses for which that health data may be utilized. Other authorizations are possible, including but not limited to what entity or entities may use the subject's health data.

A subject can provide a use right authorization in a variety of ways. According to one possible embodiment, the health data storage and access system 200 comprises or is in communication with a use right portal 280 through which a user can provide use right authorization. The portal may be accessible remotely, such as via the internet or a local network, and—after the user is authenticated—can present the subject's health data and use right options through which the user can make selections. Once provided, the use right authorization(s) can be associated with the health data for that subject. If consent is arranged offline, authorized personnel can access the portal remotely and edit use right options to reflect the patients' use right authorizations.

At step 120 of the method, the health data storage and access system 200 receives, requests, or otherwise obtains health data for a plurality of subjects. The health data can be received or obtained from any source, including from one source or from multiple sources. According to one possible embodiment, the health data storage and access system comprises or is in direct or indirect communication with an electronic medical record (EMR) database or system 270, comprising health data for a plurality of patients or subjects, and the system receives the health data from that database or system. Many other sources, including but not limited to EMR, are possible.

According to an embodiment, some or all of the health data comprises a plurality of data points for a subject. Thus, health data for a subject or patient can comprise one data point or multiple data points.

According to an embodiment, some or all of the health data comprises use right authorization granted by the subject. In other words, the subject may provide the health data storage and access system with information about use rights for that subject's health data. The use right authorization is then associated with the health data for that subject. The use right authorization controls one or both of: (i) which data points in the health data for that subject may be used; and (ii) the uses for which that health data may be utilized. Other authorizations are possible, including but not limited to what entity or entities may use the subject's health data.

According to an embodiment, examples of (i) which data points in the health data for that subject may be used include broad authorizations such as “no identifying information,” or specific authorizations such as authorization to use a diagnosis, treatment, vital signs, or any other specific authorization. However, patient names and MRN, and even date of birth, by default will not be shared. Age can be authorized in granular fashion, for example but not limited to the exact age or age range or broad age classifications. The patient has the ability to authorize at the level of each event for which data arises and to authorize all or none of the available health data. Notably, an authorization can be or comprise an instruction not to utilize data. According to an embodiment, examples of (ii) the uses for which that health data may be utilized include authorization to use health data for research, training, testing, or any other use.

A subject can provide a use right authorization in a variety of ways. According to one possible embodiment, the health data storage and access system 200 comprises or is in communication with a use right portal 280 through which a user can provide use right authorization. The portal may be accessible remotely, such as via the internet or a local network, and—after the user is authenticated—can present the subject's health data and use right options through which the user can make selections. Once provided, the use right authorization(s) can be associated with the health data for that subject.

Thus, according to an embodiment, the health data arising from each event for the subject is associated with the use right authorization provided by that subject. The health data can be associated with the use right authorization in a variety of ways, including as described or otherwise envisioned herein.

At step 130 of the method, the health data storage and access system 200 removes identifying information from the health data to generate de-identified health data. Identifying information can be removed in a wide variety of ways. For example, the system may be pre-programmed or otherwise comprise rules or a machine learning algorithm designed to recognize data that comprises identifying information, and thus remove that data to generate the de-identified health data. Many other mechanisms are possible. Most importantly, the access token is unique for each health data record that is stored, thereby preventing the creation of a proxy or a secondary patient identifier.

According to an embodiment, the de-identified health data for each subject is associated with an access token that identifies the original health data for that subject. This allows the system, or a requester of health data, to identify and/or retrieve the original health data for that subject if the system or requester is authorized to do so.

At step 140 of the method, the health data storage and access system 200 encrypts the PHI data stored in the “patient vault.” The health data can be encrypted in a variety of ways including those known in the art and developed in the future. The result of encryption will be that the health data cannot be understood or used without decryption. Accordingly, if the encrypted health data is intercepted or retrieved without access or permission, the encrypted health data will be useless.

At step 150 of the method, health data storage and access system 200 stores the encrypted PHI data in a patient data database. The encrypted PHI data can be stored in the patient data database using any method for storing encrypted data. According to an embodiment, the stored encrypted PHI data for each subject is associated in the database with: (i) a unique subject token for that subject; (ii) the use right authorization received from that subject; and (iii) the corresponding access token for the health data record.

According to an embodiment, the unique subject token for a subject is an identifier for that subject, as described or otherwise envisioned herein. According to an embodiment, the use right authorization received from that subject is the authorization received from the subject that regulates authorized use of the subject's health data, as described or otherwise envisioned herein.

According to an embodiment, the corresponding access token for that subject is the token that identifies the original health data for that subject and enables the system, or a requester of health data, to identify and/or retrieve the original health data for that subject if the system or requester is authorized to do so, as described or otherwise envisioned herein.

At step 160 of the method, the health data storage and access system 200 receives a request for access to the stored encrypted PHI data. The request can be communicated to the system via any mechanism, including from local and or remote sources. The entity or individual requesting the data is a requester, and this requester can be any entity or individual that seeks to utilize the stored encrypted PHI data. The utilization of the data can be according to any of the purposes or reasons described or otherwise envisioned herein, among others. According to an embodiment, the requester comprises or is associated with a unique requester access key. The unique requester access key is utilized by the system to determine what portion(s) of the stored encrypted PHI data the requester is authorized to access or utilize.

At step 170 of the method, the health data storage and access system 200 determines which stored encrypted PHI data can be accessed by the requester. According to an embodiment, the system determines which stored encrypted PHI data can be accessed by the requester based on the based on the unique requester access key and the use right authorizations received from the subjects. Thus, in order for a requester to receive access to health data, two conditions must be satisfied. First, the health data must be authorized, by the subject based on the use right authorization received from the subject, for use. Second, the requester must have the proper access to the authorized PHI data, based on the unique requester access key.

At step 180 of the method, the health data storage and access system 200 provides access to the requester to only the stored encrypted de-identified health data for which the requester is determined to be authorized to access. This access is based on the determination made by the system in step 170 of the method. There are many different possible mechanisms for access. According to one embodiment, all of the authorized health data is provided to the authorized requester. According to another embodiment, the authorized requester must indicate what authorized health data is to be provided, which can be fewer data elements than allowed by the patient. Other methods are possible.

Provision of the stored encrypted PHI data for which the requester is determined to be authorized to access provides a practical application of the method. Since the provided health data is de-identified when it is stored, it is of a form substantially different from the identifiable information that was originally provided to the health data storage and access system 200. In other words, all users have access to unencrypted health data for use, while only an authorized requester receives encrypted PHI data, for approved cases. Accordingly, the initial PHI separation and encryption process, the verification of use case and access rights, and the recombining of PHI data with the de-identified health data are processes that cannot be performed by a human being, and cannot be performed in the human mind, as a human mind cannot comprise data that is both unencrypted identifiable health data and decrypted PHI data. Once a human mind receives unencrypted data, there is no process for “forgetting” the identifiable health data. Thus, such a computer system is indeed capable of “forgetting” identifiable health data, since it is converted into encrypted PHI data, this computer system is performing a function that a human being, and a human mind, cannot perform.

At step 190 of the method, the health data storage and access system 200 receives a request for the original health data for one or more subjects. This request may be received together with, or after, a request from the requester for access to encrypted PHI data. The request may be provided to the system in a wide variety of different ways.

At step 192 of the method, the health data storage and access system 200 determines that the requester has authorization to access the original health data for one or more subjects. This can be done in a wide variety of different ways. According to an embodiment, the system comprises a database or table or other data structure that indicates which requesters do and/or do not have authority to access original health data. According to an embodiment, the system comprises a set of databases, rules, and algorithms to determine the use of the data and to process the data to create a limited set of PHI, such as by grouping by age ranges or by areal locations.

At step 194, the health data storage and access system 200 provides access to the recombined health data containing unencrypted de-identified data matched to the now decrypted PHI data, for the one or more subjects, for which the requester has authorization and for a direct healthcare delivery use. The system and/or requester can utilize the access token associated with each of the one or more subjects to access the original health data, as described or otherwise envisioned herein.

At step 196, if the requester has approved access, the health data storage and access system 200 processes PHI to generate limited PHI if the use case is not related to direct healthcare delivery and if the authorization matches the use case of the requester. In this way, access is provided by system 200 of unencrypted de-identified data combined with limited PHI. The system and/or requester can utilize the access token associated with each of the one or more subjects to access the original health data, as described or otherwise envisioned herein.

### Example

The following is an example of one or more components of the health data storage and access system 200. It will be understood that this is just an example of possible embodiments, and thus is non-limiting.

According to an embodiment, the health data storage and access system 200 comprises a system and method for token-based options activation, where tokens are linked to actual sales options. The tokens can be, for example, hospital specific. The tokens can be linked to an associated set of ETL and compute procedures, for a specific set of insight options. According to an embodiment, all data transformation modules have de-identification. The source application can be stored as a launch string to the patient record. This launch string can be customized to external vendor applications. And by default, a single-sign-on application launch string can be considered for certain products, although other methods are possible. User access authorization is thus controlled by the original source application. In this way, original patient data can always be accessed by authorized clinical providers.

According to an embodiment, the health data storage and access system 200 comprises a database that provides the basic architecture to hold tables designed for one or more purposes. This can be called, for example, an insights database. According to an embodiment, each data model, supporting key insight areas such as operations, clinical, and research-scientific use, is designed to work in a self-sufficient manner. According to an embodiment, relationships between the data tables in the insights database comprise a “unified data model.” The relationships between the data tables in the insights database can be enabled for purchased or authorized options, and the tables can be linked together without dependencies and can fit together like a set of bricks. According to an embodiment, each option—which can be comprised of its own transformation and compute modules, and its data structure—can be improved independently.

According to an embodiment, the health data storage and access system 200 comprises a data control element, which can be called a “Patient Data Use Exchange” or PDUE. As part of each data element being loaded into the insight database, PHI is removed. However, PHI is stored in an encrypted “patient vault” and tokens are created to do the following:

According to an embodiment, one token can be an access string invoking the source application. Many vendors have external single sign-on capabilities, usually via a URL. Any such activation string can be placed here. In this way, de-identified data can be resolved back to the original patient record and the now re-identified data can be made available for authorized users.

According to an embodiment, the Patient Data Use Exchange has additional properties. For example, it can track these use rights parameters to (but not limited to): (i) an application based data use right, including default use (standard of care; clinical quality), research, new algorithm development, 3^{rd} party tools, opt-in to artificial diagnostic technologies, and more; (ii) data use conditions including payment to patient, credits, and more; (iii) age, including at time of study; hidden, binned numerical, summary text, raw, and more; (iv) gender—self-identified (or unanswered); (v) ethnicity-self-identified (or unanswered); and (vi) EventDate, among other use rights parameters. As a part of the design of the PDUE, no patient names, MRNs, street address, or age will be available by default. Numerical values and other quasi-identifiers can be identified and processed to further aggregate and obscure specific patient identifiers (for example but not limited to using “adult” or “25-45” to categorize age and to use an aggregate label to create groupings of zip codes).

According to an embodiment, these parameters and tokens are stored in the Patient data use exchange. Access states are converted into public-private key tokens.

According to an embodiment, the Data use exchange has the capability to serve as a broker for access and use right execution. For example, patient data can be processed according to parameters in the Use Exchange, an access token can be generated for each vendor, and patient data can be signed by a unique token and the access token. According to an embodiment, when data access request is made, the access key is provided by the vendor. A calculation is made to identify vendor keys that resolve Patient's key. If the patient's key is returned, then authorization is approved. Rather than a Boolean, in this way, the Use Exchange acts as a gateway for authentication and authorization. This also enables the use of public-key encryption to actually transfer the data.

TABLE 1
Example Patient Data Use Exchange

| PatientToken | Event | Property | Value |
|---|---|---|---|
| 0x0284Ge | 1xd332ga | URL platform | iecg . . . (ss0) |
| 0x0284Ge | 1xd332ga | use right | research, vendor |
| 0x0284Ge | 1xd332ga | age | binned |
| 0x0284Ge | 1xd332ga | Gender | do not use |
| 0x0284Ge | 1xd332ga | race/ethnicity | do not use |
| 0x0284Ge | 1xd332ga | Token-platform1 | 9xabfgf88 |
| 0x0284Ge | 1xd332ga | Token-platform2 | Gxlaad100 |
| 0x0284Ge | 1xd332ga | EventDate | Jan. 1, 2024 |
| Fx3cc1092 | 0x2127ab | URL platform | iscv . . . (sso) |
| Fx3cc1092 | 0x2127ab | use right | research, vendor |
| Fx3cc1092 | 0x2127ab | age | Adult |
| Fx3cc1092 | 0x2127ab | gender | M |
| Fx3cc1092 | 0x2127ab | race/ethnicity | Caucasian |
| Fx3cc1092 | 0x2127ab | EventDate | Mar. 1, 2024 |
| Fx3cc1092 | 0x2127ab | Token-platform4 | Gx10abcca |
| Fx3cc1092 | 0x2127ab | Token-platform2 | 6x617289a |

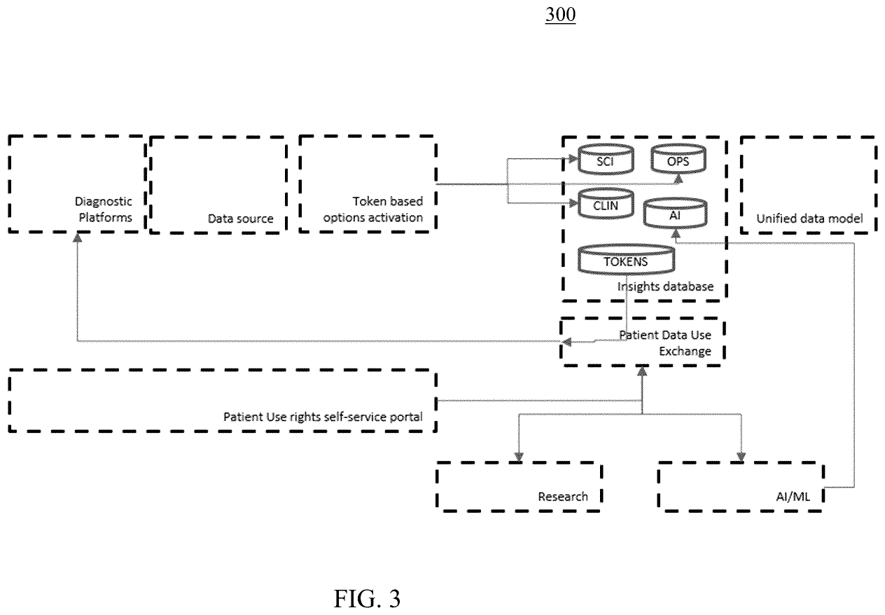

Referring to FIG. 3, in one embodiment, is a flowchart 300 of a method for storing and controlling access to de-identified health data for a plurality of subjects.

According to an embodiment, the basic architecture depends on the nature of the source platform installed. Currently, most diagnostic platforms reside on-premise at a hospital, although vendors continue to be ready for cloud deployment. One embodiment is that the application is deployed on hospital data warehouse/cloud, and the analytics infrastructure is hosted on a vendor cloud. The implementation can be cross-cloud platforms as a result.

In another embodiment, the Patient Data Use Exchange serves in the role of a patient data model coordinator (usually in the form of a lookup index table, for all tracked events and date, by patient. In this way, the PDUE coordinates among different results: from the clinical, operations, and scientific modules, from research modules from the hospital, and from third-party ML/AI results (for example Philips with Anumana ML-plugins). In this way, the PDUE gates and tracks bi-directional data flow (patient data to algorithm developers and storing results from third-party algorithms). In additional to conventional patient flow through the hospital, the PDUE can track encounters with algorithms as well. PDUE access is controlled by hospital policy, and can be consulted in the form of an application programming interface. The API can resolve and return available data, with additional policy restrictions as set by the hospital. Data can be encrypted in-flight and sent to the requesting application.

Another embodiment is that ETL, compute, and data structures are implemented as serverless instances and processes, rather than as cloud hosted servers.

In another embodiment, a patient data use portal can be used to track data flows for hospital use and partner use, which can be external to the hospital. This can be a simple deployment without the need for encryption key use. This can be leveraged if the entire embodiment works within the cloud, where data-in-flight and data-at-rest security are provided by the certified cloud provider. A similar situation exists if the application is hosted within hospital firewall, allowing for simple lookup of data use rights without any encryption capability. According to another embodiment, the system provides the capability to secure data-in-flight, should the data pass out of hospital firewalls.

Referring again to FIG. 2 is a schematic representation of a health data storage and access system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 comprises or is in direct or indirect communication with an electronic medical record (EMR) database or system 270, comprising health data for a plurality of patients or subjects. The health data can be any information about the plurality of patients or subjects. According to an embodiment, the information comprises one or more of demographic information about the patient, a diagnosis for the patient, medical history of the patient such as treatment information, and/or any other information.

According to an embodiment, system 200 comprises or is in direct or indirect communication with a use right portal 280 through which a user can provide use right authorization. The portal may be accessible remotely, such as via the internet or a local network, and—after the user is authenticated—can present the subject's health data and use right options through which the user can make selections. Once provided, the use right authorization(s) can be associated with the health data for that subject.

According to an embodiment, storage 260 of system 200 may store one or more algorithms, modules, and/or instructions to carry out one or more functions or steps of the methods described or otherwise envisioned herein. For example, storage 260 may comprise, among other instructions or data, PHI removal instructions 262, encryption instructions 263, and/or authorization instructions 264.

According to an embodiment, PHI removal instructions 262 direct the system to remove identifying information from received health data to generate de-identified health data and to store PHI data into the patient vault. Identifying information can be removed in a wide variety of ways. For example, the system may be pre-programmed or otherwise comprise rules or a machine learning algorithm designed to recognize data that comprises identifying information, and thus remove that data to generate the de-identified health data. Many other mechanisms are possible.

According to an embodiment, encryption instructions 263 direct the system to encrypt the PHIdata. The health data can be encrypted in a variety of ways including those known in the art and developed in the future. The result of encryption will be that the health data cannot be understood or used without decryption. Accordingly, if the encrypted health data is intercepted or retrieved without access or permission, the encrypted health data will be useless.

According to an embodiment, authorization instructions 264 direct the system to determine which stored encrypted PHI data can be accessed by the requester. According to an embodiment, the system determines which stored encrypted PHI data can be accessed by the requester based on the based on the unique requester access key and the use right authorizations received from the subjects. Thus, in order for a requester to receive access to health data, two conditions must be satisfied. First, the health data must be authorized, by the subject based on the use right authorization received from the subject, for use. Second, the requester must have the proper access to the authorized health data, based on the unique requester access key.

According to an embodiment, authorization instructions 264 also direct the system to 200 determine whether a requester has authorization to access the original health data for the one or more subjects. This can be done in a wide variety of different ways. According to an embodiment, the system comprises a database or table or other data structure that indicates which requesters do and/or do not have authority to access original health data.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for storing and controlling access to protected health information (PHI) data for a plurality of subjects, comprising:
obtaining health data for the plurality of subjects, wherein the health data comprises one or more data elements for each of the plurality of subjects, and wherein the health data comprises, for at least some of the plurality of subjects, use right authorization received from the subject;
removing identifying information from the health data to generate de-identified health data and PHI data, wherein the de-identified health data for each subject is associated with an access token that identifies the original health data for that subject;
encrypting the PHI data;
storing the encrypted de-identified health data in a patient data database, wherein the stored encrypted PHI data for each subject is associated with: (i) a unique subject token for that subject; (ii) the use right authorization received from that subject; and (iii) the corresponding access token for that subject;
receiving, from a requester, a request for access to the stored encrypted PHI data, wherein the requester comprises a unique requester access key;
determining, based on the unique requester access key and the use right authorizations received from the subjects, which stored encrypted PHI data can be accessed by the requester; and
providing, to the requester based on the determination, access to only the stored encrypted PHI data for which the requester is determined to be authorized to access.

2. The method of claim 1, wherein the encrypted PHI data for each subject is stored as a plurality of data elements, each of the plurality of data elements associated with (i) the unique subject token for that subject; (ii) the use right authorization received from that subject; and (iii) the corresponding access token for that subject.

3. The method of claim 1, further comprising the step of receiving, from a subject, use right authorization for the subject's health data.

4. The method of claim 3, wherein the subject accesses a remote portal to provide the use right authorization.

5. The method of claim 1, further comprising the steps of:
receiving, from a requester, a request for the original health data for one or more subjects;
determining that the requester has authorization to access the original health data or PHI data for the one or more subjects;
providing, using the access token associated with each of the one or more subjects, access to the original health data for the one or more subjects for a use case related to healthcare delivery; and/or providing, using the access token associated with each of the one or more subjects, limited and aggregated PHI data for the one or more subjects, for a use case not related to healthcare delivery.

6. The method of claim 1, wherein the use right authorization received from the subject comprises which data points in the health data for that subject may be used.

7. The method of claim 1, wherein the use right authorization received from the subject comprises one or more uses for which that health data may be utilized.

8. The method of claim 1, wherein the use right authorization received from the subject comprises an identification of one or more entities authorized to use the subject's health data.

9. A system for storing and controlling access to protected health information (PHI) data for a plurality of subjects, comprising:
health data for the plurality of subjects, wherein the health data comprises one or more data elements for each of the plurality of subjects, and wherein the health data comprises, for at least some of the plurality of subjects, use right authorization received from the subject;
a processor configured to: (i) remove identifying information from the health data to generate de-identified health data and PHI data, wherein the de-identified health data for each subject is associated with an access token that identifies the original health data for that subject via the patient vault; (ii) encrypt the PHI data; (iii) store the encrypted PHI data in a patient data database, wherein the stored encrypted PHI data for each subject is associated with: (1) a unique subject token for that subject; (2) the use right authorization received from that subject; and (3) the corresponding access token for that subject; (iv) receive, from a requester, a request for access to the stored encrypted PHI data, wherein the requester comprises a unique requester access key; (v) determine, based on the unique requester access key and the use right authorizations received from the subjects, which stored encrypted PHI data can be accessed by the requester; and (vi) provide, to the requester based on the determination, access to only the stored encrypted PHI data for which the requester is determined to be authorized to access.

10. The system of claim 9, wherein the encrypted PHI data for each subject is stored as a plurality of data elements, each of the plurality of data elements associated with (i) the unique subject token for that subject; (ii) the use right authorization received from that subject; and (iii) the corresponding access token for that subject.

11. The system of claim 9, wherein the processor is further configured to receive, from a subject, use right authorization for the subject's health data.

12. The system of claim 9, further comprising an authorization portal.

13. The system of claim 9, wherein the use right authorization received from the subject comprises which data points in the health data for that subject may be used.

14. The system of claim 9, wherein the use right authorization received from the subject comprises one or more uses for which that health data may be utilized.

15. The system of claim 9, wherein the use right authorization received from the subject comprises an identification of one or more entities authorized to use the subject's health data.
